(19) United States
(12) Patent Application Publication
(10) Pub. No.: US 2006/0247504 A1
(43) Pub. Date: Nov. 2, 2006

(54) Residential monitoring system for selected parameters

(75) Inventor: Lee D. Tice, Bartlett, IL (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International, Inc., Morristown, NJ (US)

(21) Appl. No.: 11/119,182

(22) Filed: Apr. 29, 2005

(51) Int. Cl. A61B 5/00; A61B 5/04

(52) U.S. Cl. 600/300; 128/903; 600/388

(57) ABSTRACT

A monitoring system for one or more physiological conditions includes at least one sensor couplable to an individual whose condition is being monitored. The sensor is in real-time wireless, continuous communication with a displaced unit, which could be local to the individual. The displaced unit evaluates incoming signals from the sensor to determine if the individual or other caregivers need feedback as to a sensed condition.

### FIELD OF THE INVENTION

The invention pertains to residential monitoring systems. More particularly, the invention pertains to such systems which can continually monitor selected parameters of an individual such as blood pressure, respiration rate, heart rate, temperature or the like.

### BACKGROUND OF THE INVENTION

Various known in-residence monitoring systems are oriented around an individual taking routine medications or performing routine activities. The individual will normally go to a central location within the residence and attach sensors associated with the one or more physiological parameters to be measured. These systems provide for occasional comprehensive physiological monitoring in remote settings. However, such systems are generally limited in their capabilities and do not provide for fast response in emergency situations. They are relying upon the condition of the individual being relatively stable between the times of physiological measurement. These systems are basically trying to replace doctor visits with in-residence monitoring of the same physiological parameters that the doctor would monitor in his office. This saves time and cost for the individual and doctor.

A remote location may have a monitoring station that can receive and send information to a multitude of patients. A nurse or patient monitor may review the information transmitted from the in-residence system to determine if the individual is taking his/her medications or if the scheduled measurements of their physiological parameters require a personal visit to the residence.

The equipment used in these systems if generally bulky and uses hard wiring between the physiological sensors and the system. There is a need to make the physiological sensing more flexible and portable to allow the patient more movement within the residence without losing the monitoring capabilities.

It would be desirable to combine communications protocols and technological advances in sensing and monitoring equipment to provide life safety monitoring features in residential monitoring systems. Electronics are becoming more compact and energy efficient. Communications technologies can provide reliable two-way communication links with relatively low power. Preferably, equipment which benefits from such trends could be utilized to provide light, low power wireless sensors that could be worn to provide real-time information as to the condition of the respective individual.

### BRIEF DESCRIPTION OF THE DRAWINGS

FIG. 1 is an overall view of a system in accordance with the invention;

FIG. 2 is a block diagram of sensor assemblies usable in the system of FIG. 1; and

FIG. 3 is a block diagram of a base unit usable in the system of FIG. 1.

### DETAILED DESCRIPTION OF THE EMBODIMENTS

While this invention is susceptible of embodiment in many different forms, there are shown in the drawing and will be described herein in detail specific embodiments thereof with the understanding that the present disclosure is to be considered as an exemplification of the principles of the invention and is not intended to limit the invention to the specific embodiments illustrated.

Methods and systems which embody this invention use sensors in a continual monitoring mode. The continual sensing of physiological parameters of an individual in a residence while allowing movement of the individual provides a significant improvement in the quality of life of the individual. Such systems and methods can immediately detect any abnormalities that require immediate response to the condition of the individual. In addition, they can provide immediate feedback to the individual when their activities become life threatening or if they are not taking proper care of themselves. For example, if their blood pressure is dropping or some other measured parameter indicates that the individual should take some immediate action to prevent an emergency situation, then that individual becomes aware and can prevent it prior to it happening.

In accordance with the invention, this continual monitoring of selected parameters combines battery operated sensors, battery condition monitors, and a wireless link with an in-residence unit. Monitoring can be carried out in real-time.

Preferably, the sensors will be easy to couple to the individual. In accordance with the invention, one way of doing this is to at least partially embed the sensors into some apparatus or clothing that the person wears. For example, if the sensors are embedded into an elastic type undershirt that the person wears, sensor contacts will engage the body as soon as the person wears the shirt. Instead of an undershirt, any elastic apparatus or garment fitting to the body could be used. If electrical enhancement compounds or solutions are needed for electrical contact with the body, they could be applied as needed or incorporated into the sensing pads for connection. It is possible that this elastic type garment could be worn for several days to provide continuous sensing, even during sleep. When this elastic type of garment or member is removed, its replacement will incorporate the sensors or at least the sensor contacts. The sensors can be separately attached.

If the person is in a wheel chair, then the sensors could be incorporated into the wheel chair with quick connect/disconnect attachments to the person to allow them to leave the wheel chair for using bathroom facilities. The battery, battery monitoring, sensing circuits, and wireless communications can be built in or attached to the wheel chair.

Some examples of sensors that can be incorporated for continual monitoring include heartbeat, blood pressure, oxygen level, breathing rate, body temperature, movement, perspiration or skin conductivity, swelling of limbs such as legs and feet. Other types of sensors could be used which are responsive to a physiological problem or problems being monitored,

Such continual monitoring could alert the person to a problem or communicate a need for an intermediate intervention before it becomes life threatening. The person could also take action to rectify the situation if it involves activities within his/her means. For example, if a perspiration sensor is indicating that the person is dehydrated, then the system could respond to the person to encourage him/her to consume enough water.

Additionally, a system that embodies the invention, can record information on a continual, real-time, basis to provide a record of the resident. This information can be communicated to a remote location and stored. It can be used to determine if there are some patterns during the day or night for abnormalities in measurement or if the individual's condition appears to be normal. For example, if a physiological measurement becomes abnormal at certain times of the day, the monitoring person at a remote location can forward that information to a doctor to determine if medications are properly working or prescribed at the correct times for that resident.

As residents age, they may need more guidance on a continual basis. Additional guidance can be provided for the well being of the resident.

The charge status of the batteries in such systems will preferably be monitored.

If the resident is capable of changing batteries, then the system can monitor the performance of that task. The system can provide procedures and information to the resident for replacing of the batteries with allotted times for the tasks.

In addition, information as to battery charge status can be transmitted to a remote monitoring location. This information may contain data regarding the need to order more batteries for replacement or charging of rechargeable batteries.

If rechargeable batteries are being removed but not put into the charger, someone from the remote monitoring facility can call the resident with a reminder to place the removed batteries into a charger. Alternatively, the local system can communicate this information to the resident. When completed by the resident, the charger can send a signal that it is functioning properly with a battery under charge.

In a preferred embodiment, a system that embodies the invention incorporates portable battery operated sensors and associated circuitry, portable battery operated wireless transmitters, and portable battery packs with charge monitoring circuitry worn by the resident. Each sensor assembly contains all the equipment necessary to transmit that sensor's information. There are no wires going between the sensors and a centralized battery source on the person. This means that if a transmitter fails, the system only loses the associated sensor(s) and remaining sensors continue to operate. This also makes the use of the sensors easier since no wires will become tangled.

The sensors and associated circuitry interface to the wireless transmitter to periodically transmit information to another location which may be local or remote or both. If the wireless transmitter incorporates cell phone dialing and information exchange features, then the remote location could receive and transmit information directly from or to that transmitter.

In summary, the invention is directed to the improvements in the portability of sensors and in establishing radio frequency or other wireless communications between the sensors and the equipment used for communication of information to and from remote locations outside the residence.

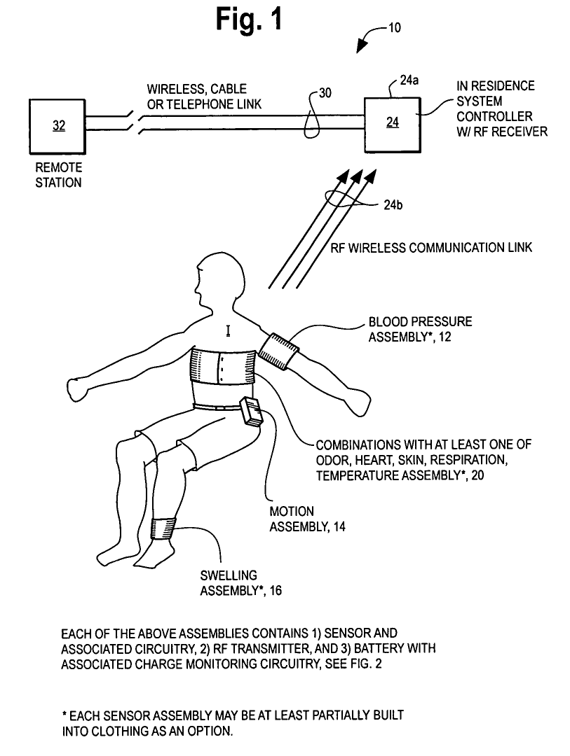

FIG. 1 illustrates system 10 in accordance with the present invention. An Individual I can be equipped with a variety of wireless sensor combinations to monitor one or more selected physiological conditions. Each of the sensor combinations could be self contained and couplable to the individual I using elastic bands, self-attaching straps, or the like without limitation. Alternately, they may be at least partly built into clothing worn by the Individual.

Representative sensor assemblies include a blood pressure assembly 12, a motion sensing assembly 14, an assembly 16 to sense swelling of one or both of the lower extremities of the Individual. Additionally, on one or more sensor combinations including, for example, a heart monitor, a skin sensor, a respiration sensor, temperature sensor, and/or odor sensor assembly 20 could also be provided and coupled to the chest area of the Individual.

The various sensor combinations 12 . . . 20 are preferably in real-time wireless communication with a local base or monitoring unit 24. The monitoring unit can continually receive wireless, for example RF, signals from the respective sensor assemblies 12 . . . 20 to keep track of cardiac, respiratory function, temperature and the like all without limitation. The local station 24 can be implemented with a programmable processor and software, discussed subsequently to a screen or analyze incoming RF signals from the various sensor assemblies 12 . . . 20 to continually monitor the ongoing condition of the Individual.

In the event that an abnormality is sensed, a signal can be sent via medium 30 to a remote station 32 for consideration by a trained professional for follow-up. Alternately, the local device 24 can notify a neighbor or other person available in the area who could respond quickly to check on the condition of the Individual. Abnormal conditions which can be responded to both locally and with messages to the remote station 32 include cardiac events, respiratory failure, temperature variations and the like all without limitation. Thus, the system cannot only continually monitor physiological conditions which are exhibiting normal parameter ranges, but it can also provide immediate follow-up for unexpected conditions.

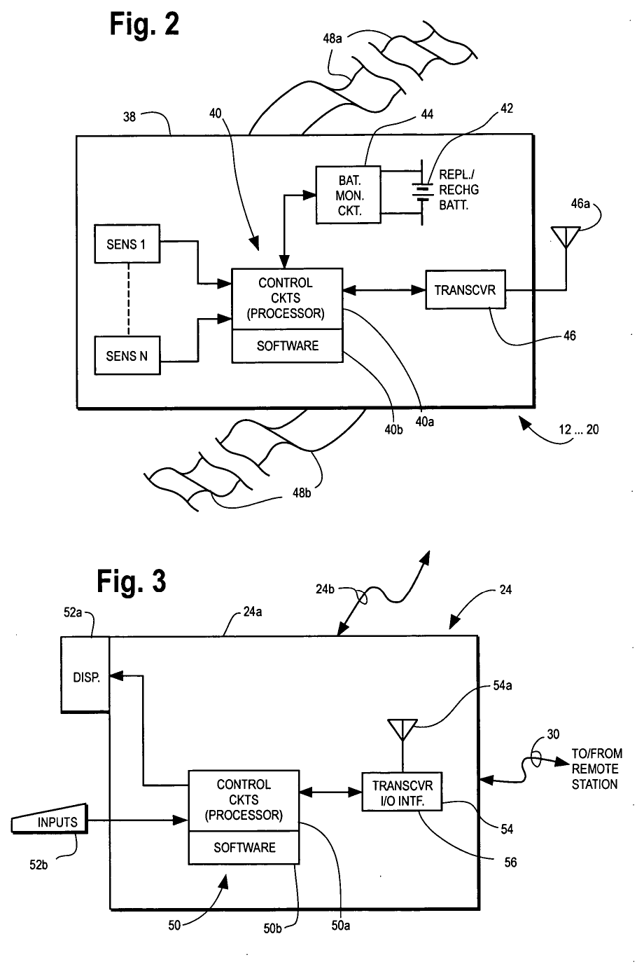

FIG. 2 is a block diagram of a representative one of the sensor assemblies 12 . . . 20. The respective assembly includes a housing 38 which in one embodiment carries one or more sensors SENS1 . . . N which respond to a selected physiological condition. Outputs from the respective sensor(s) SENSI are coupled to a local control circuitry 40 which could be implemented at least in part with a programmable processor 40a controlled by local software 40b. The respective assembly includes a replaceable battery 42, which could be rechargeable, and battery monitoring circuitry 44. The assembly also includes an input/output circuitry which includes a transceiver 46 which could at least transmit RF signals via the antenna 46a to the local base unit 24. The sensor assembly 12 . . . 20 could in fact be in bi-directional wireless communication with the local unit 24 if desired. The sensor assemblage 12 . . . 20 could be coupled to the Individual I via elastic or self-attaching straps such as 48a, b all without limitation. Alternately, portions of the sensor assemblage 12 . . . 20 could be incorporated into clothing worn by the Individual I with connectors being provided to couple signals between those portions of the sensor assembly carried on the Individual's body and those portions carried on the adjacent clothing.

FIG. 3 is a block diagram of the local or base station 24. One known form of a local or base station has been disclosed and described in U.S. patent application Ser. No. 10/956,681 filed Oct. 1, 2004 and entitled “Global Telephonic Device and Base Station” which has been assigned to the Assignee hereof. The '681 application is incorporated herein by reference.

The base station 24 includes the housing 24a which carries control circuitry 50 which could be implemented at least in part by a programmable processor 50a and associated prestored software 50b. The control circuitry 50 can communicate via a local display 52a and can receive manually entered inputs by a keyboard or switches 52b. Further, the base unit 24 includes at least a receiver, preferably a transceiver 54 for communicating wirelessly 24b via an antenna 54a with the sensing assemblies 14 . . . 20. An input/output interface 56 is also provided enabling the unit 24 to communicate via the median 30 with the remote station 32. It would be understood that the median 30 could be any selected wired wireless median without limitation.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

What is claimed:

1. A system comprising:
a plurality of sensor assemblies, each assembly includes at least one sensor of a selected physiological condition of an individual, control circuitry coupled to the sensor, and, a wireless interface coupled to the control circuitry, for at least transmitting sensor provided information relative to the respective condition to a displaced unit substantially in real-time where each assembly includes an attachment structure for attaching at least part of the respective assembly to the person.

2. A system as in claim 1, where each assembly includes at least a wireless transmitter.

3. A system as in claim 1, where the displaced unit includes a receiver of wireless signals from the assemblies and control software coupled thereto for evaluating received information relating to at least one sensed physiological condition to determine if immediate attention is required.

4. A system as in claim 3 which includes software at the displaced unit, responsive to a determination that immediate attention is required, for initiating communication with a remote unit.

5. A system as in claim 1, where at least some of the attachment structures include at least one of elastic material, first and second members which overlap and releasibly engage one another, or, an adhesive member.

6. A system as in claim 1, where at least some of the assemblies have a sensor part which carries the at least one sensor and an electrical part which carries at least the control circuitry, the two parts being electrically couplable together.

7. A system as in claim 6, where the sensor part carries the attachment structure.

8. A system as in claim 7, where the electrical part is carried by an article of clothing wearable by the person.

9. A system as in claim 8, where the article of clothing corresponds to at least one of a shirt, or pants.

10. An in-residence monitoring system comprising:
at least one portable sensor transportable by a person for sensing at least one physiological condition of the person and an interface for wirelessly transferring data to at least one of an in-residence device or personal computer, where the in-residence device or personal computer includes at least one of an indicator or display;
where the at least one portable sensor is powered by a battery, circuitry to monitor the battery for remaining energy level relative to a predetermined threshold level for the purpose of at least one of indicating immediate battery replacement or scheduling battery replacement;
where the in-residence device or personal computer includes a processor and software to at least in part analyze data from the sensor to determine the existence of a condition of the person requiring an immediate response;
in response to at least one of a determined condition requiring an immediate response or the battery being below a predetermined energy level, the in-residence device or personal computer transfers data to at least one displaced unit.

11. A system as in claim 10, where the processor includes a memory.

12. A system as in claim 10, where the portable sensor includes at least one sensor partially imbedded at least in part in the clothing of a person being monitored such that putting the clothing onto the person establishes, at least in part, sensing of the person.

13. A system as in claim 10, where the portable sensor is substantially continually sensing at least one physiological condition.

14. A system as in claim 10, where the portable sensor is at least one of a respiration sensor, cardiac sensor, body temperature sensor, perspiration sensor, blood oxygen level sensor, skin color sensor, blood pressure sensor, odor sensor, smoke sensor, wound sensor, pressure sensor, swelling sensor, or movement sensor.

15. A system as in claim 12, where the clothing is at least in part elastic.

16. A system as in claim 10, where the displaced unit includes at least one of a telephone or a computer.
